(19) United States
(12) Patent Application Publication
(10) Pub. No.: US 2006/0127447 A1
(43) Pub. Date: Jun. 15, 2006

(54) Poly-gamma-glutamate having ultra high molecular weight and method for using the same

(76) Inventors: Moon-Hee Sung, Seoul (KR); Chung Park, Daejeon (KR); Seung-Pyo Hong, Daejeon (KR); Kwang Seok Kim, Daejeon (KR); Jae Jun Song, Daejeon (KR); Eu Gene Rha, Daejeon (KR); Kwang Kim, Daejeon (KR); Kenji Soda, Kyoto (JP); Makoto Ashiuchi, Kochi (JP); Ha Ryoung Poo, Daejeon (KR); Seung Goo Lee, Daejeon (KR)

Correspondence Address: INTELLECTUAL PROPERTY / TECHNOLOGY LAW, PO BOX 14329, RESEARCH TRIANGLE PARK, NC, 27709, US

(21) Appl. No.: 10/520,557

(22) PCT Filed: Jul. 10, 2003
(86) PCT No.: PCT/KR03/01369

§ 371 Date: Jan. 6, 2005

(30) Foreign Application Priority Data
Jul. 10, 2002 (KR) 10-2002-0040083

(51) Int. Cl. A61K 38/16; A61K 47/00; C07K 14/32; C12N 1/21; C12P 21/06

(52) U.S. Cl. 424/439; 514/12; 530/350; 435/69.1; 435/252.31

(57) ABSTRACT

The present invention relates to a poly-gamma-glutamate (PGA) having an ultra-high molecular weight greater than 5,000 kDa. The ultra-high molecular weight PGA according to the present invention has a mean molecular weight greater than 13,000 kDa, and more than 95% of its molecules have a molecular weight ranging from 3,000 to 15,000 kDa. Also, it can be produced by the culturing of Bacillus subtilis var. chungkookjang. The ultra-high molecular weight PGA according to the present invention shows very excellent moisture-absorbing, moisture-retaining, sustained release, mineral solubility, and water-absorbing properties, and thus, can be used as a new and high value-added material in various applications.

### FIELD OF THE INVENTION

The present invention relates to an ultra-high molecular weight poly-gamma-glutamate (hereinafter, referred to as “PGA”) produced by a halotolerant strain Bacillus subtilis var. chungkookjang (KTCT 0697BP) isolated from chungkookjang, Korean traditional fermented soybean food, and also to the method of use thereof. More particularly, the present invention relates to a PGA with a molecular weight greater than 5,000 kDa showing edibility, water solubility, an anionic property and biodegradability, and also to foods, cosmetics, feedstuffs, mineral absorption-promoting compositions, which contain the same.

### BACKGROUND ART

PGA is a viscous polymer where D,L-glutamate is polymerized trough gamma-glutamyl. It is produced from a Bacillus sp. strain, which is isolated from chungkookjang as Korean traditional food obtained from the fermentation of soybeans using ricestraw, natto as Japanese traditional fermented soybean food, and kinema as Nepalese traditional fermented soybean food.

The PGA produced from the Bacillus sp. strain is a polymer having edibility, water solubility, an anionic property and biodegradability, and can be used as a raw material of moisture-absorbing agents, moisture-retaining agents and cosmetics, and a raw material for the preparation of naturally degradable plastics using the synthesis of ester derivatives.

Recently, with respect to the production and use of the PGA, there are being actively conducted studies on the development of a material as a substitute for difficultly degradable polymers, and the production of heat-resistant plastics by esterication, and the production of water-soluble fibers and membranes, etc., in highly developed countries as a leader. Furthermore, studies on a change in physical properties of the PGA occurring upon irradiation of the PGA with gamma rays, and studies on the development and industrial application of a PGA hydrogel using crosslinkers.

The PGA hydrogel is an environment-friendly material, which is produced by the intermolecular or intramolecular crosslinking of the PGA, a biopolymer produced by the culturing of Bacillus subtilis var chungkookjang, and has a water-absorbing property, biodegradability and thermoplasticity. Methods for the crosslinking of the PGA include irradiation with radiation, such as gamma rays or electron beams, treatment with chemical crosslinkers, such as epoxy resin, and the like. When aqueous PGA solution is irradiated with radiation, the crosslinking between PGA molecules takes place, thereby giving PGA resin having a water-absorbing property, biodegradability and thermoplasticity.

In the prior art, there were reported a study on an effect of manganese ions on the composition and production of PGA, a study on the production of the PGA having water solubility by ultrasonic decomposition, and a study on the production of plastics of low water solubility by synthesis with ester derivatives (Biosci. Biotechnol. Biochem., 60(8):1239-42, 1996), a study on the production of PGA using Bacillus subtilis, and a study on the use of the PGA for healthy foods having a therapeutic effect of osteoporosis, such as a calcium-dissolving agent, etc. (Japanese patent laid-open publication No. Heisei 6-32742).

In addition, there was reported an effect of PGA on the reduction of water contamination according to the reduction of a phosphorus content in a water system (European patent No. 838160). Moreover, highly gelling, water-soluble, biodegradable and adsorbent PGA resins, and the use thereof for sanitary products and foods and in horticultural industries, etc., were disclosed (Japanese patent laid-open publication Nos. Heisei 10-251402, 7-300522 and 6-322358).

Furthermore, there were known the use of PGA for solid biodegradable fibers, films or film-shaped materials by the dissolution, precipitation and drying of the PGA (Japanese patent laid-open publication Nos. Heisei 7-138364 and 5-117388), and the use of the PGA for a drug carrier (Japanese patent laid-open publication Nos. Heisei 6-92870 and 6-256220).

Meanwhile, there were known inventions on the efficient production of the PGA (Korean patent application No. 1997-67605), the production of high concentration PGA (Korean patent application No. 2001-0106025), and halotolerantstrain Bacillus subtilis var. chungkookjang of producing a high-molecular weight PGA (PCT application No. PCT/KR01/01372 corresponding to Korean patent laid-open publication No. 2001-78440).

The molecular weight of PGAs produced in the prior art is in the range of about 100-2,000 kDa, and they have limitations on the application thereof, particularly in cosmetic or food fields, in terms of the solubility, absorption and sustained release of minerals.

Accordingly, the present inventors have conducted extensive studies in an attempt to produce an ultra-high molecular weight PGA, and consequently, found that the batch culturing of Bacillus subtilis var. chungkookjang in medium containing glucose, citric acid and glutamate yielded a PGA having a molecular weight greater than 5,000 kDa without byproducts, and the produced PGA showed a very excellent effect upon the use thereof for moisture-retaining agents, water-absorbing agents, and mineral absorption-promoting agents. On the basis of this point, the present invention was perfected.

### DISCLOSURE OF INVENTION

Therefore, a main object of the present invention is to provide a PGA having an ultra-high molecular weight greater than 5,000 kDa.

Another object of the present invention is to provide cosmetics, foods and feedstuffs containing the ultra-high molecular weight PGA.

Still another object of the present invention is to provide a hydrogel produced from the ultra-high molecular weight PGA, as well as a moisture-absorbing or water-absorbing agent containing the same.

Yet another object of the present invention is to provide a mineral absorption-promoting composition, which contains the ultra-high molecular weight PGA and a mineral.

To achieve the objects as described above, the present invention provides an ultra-high molecular weight PGA having a mean molecular weight greater than 5,000 kDa.

Preferably, the molecular weight of the PGA according to the present invention is in the range of 5,000 to 15,000 kDa.

Since the PGA according to the present invention has ultra-high molecular weight, it has very excellent moisture-absorbing and moisture-retaining properties as compared to the prior PGA with relatively low molecular weight. Thus, the present invention also provides foods, cosmetics and feedstuffs containing the ultra-high molecular weight PGA.

A hydrogel produced from the PGA of the present invention as a raw material has a very excellent water-absorbing property as compared to the prior product with relatively low molecular weight. Thus, the present invention also provides a hydrogel produced from the ultra-high molecular weight PGA, as well as a moisture-absorbing or water-absorbing agent containing the same.

The PGA according to the present invention has a very excellent property of enhancing the solubility of mineral ions, and an excellent property on the sustained release of mineral ions. Thus, the present invention also provides a mineral absorption-promoting composition, which contains the ultra-high molecular weight PGA and a mineral.

In the present invention, the mineral is preferably Ca, Fe, Mg, Zn, Cu or Se, but minerals essential for a living body may also be used without special limitation.

In the present invention, the PGA may also be substituted with a copolymer of a PGA having an ultra-high molecular weight greater than 5,000 kDa and a polyamino acid bearing a positive charge. The polyamino acid is preferably polylysine or polyarginine. The PGA according to the present invention bears a negative charge, and thus, can electrostatically bind to the polyamino acid to form a copolymer.

Furthermore, the present invention provides a method for using the ultra-high molecular weight PGA with a molecular weight greater than 5,000 kDa, for a mineral absorption-promoting agent

In the present invention, the ultra-high molecular weight PGA is produced by microbial culturing. A microorganism used for the production of the ultra-high molecular weight PGA in the present invention is Bacillus subtilis var. chungkookjang (KCTC 0697BP) whose isolation, identification and physiological characteristics are described in detail in PCT application No. PCT/KR01/01372, which was filed in the name of the present inventors on Aug. 11, 2001.

The morphological and physiological characteristics of this strain are as follows.

This strain is gram-positive bacteria, which form milky colonies upon culturing on an LB agar plate, and show active growth in aerobic conditions above 37° C. and slow growth at a culturing temperature higher than 55° C. Furthermore, this strain is a halotolerant stain that can grow even at a salt (NaCl) concentration of 9.0%, which is higher than the salt tolerance of general Bacillus subtilis species. Also, it is a typical Bacillus strain, which forms endospores when it is cultured in LB liquid medium or solid medium for at least 70 hours. The comparative analysis of the 16S rDNA sequence of this strain and the 16S rDNA sequence of the prior Bacillus sp. strain reveals that this strain has a very high homology of 99.0% with Bacillus subtilis.

### BRIEF DESCRIPTION OF DRAWINGS

The above and other objects, features and advantages of the present invention will be more clearly understood from the following detailed description taken in conjunction with the accompanying drawings, in which:

FIG. 1 is a graph showing the molecular weight distribution of the PGA according to the present invention;

FIG. 2 is a graph showing the comparison between the water-absorbing property of the ultra-high molecular weight PGA of the present invention and a product of the prior art;

FIG. 3 is a graph showing the comparison between the moisture-retaining property of the ultra-high molecular weight PGA of the present invention and a product of the prior art;

FIG. 4 is a graph showing an effect of the ultra-high molecular weight PGA according to the present invention on the improvement of Ca solubility;

FIG. 5 shows a change in intestinal Ca absorption according to time, when PGA with a 5,000-kDa molecular weight of the present invention is used; and

FIG. 6 is a graph showing an effect on water absorption of a hydrogel produced from the ultra-high molecular weight PGA of the present invention as a raw material.

### DETAILED DESCRIPTION OF THE INVENTION

The present invention will hereinafter be described in further detail by examples. It should however be borne in mind that these examples are given for illustrative purpose only and the scope of the present invention is not limited to or by the examples.

Although the production of the ultra-high molecular weight PGA using Bacillus subtilis var. chungkookjang (KCTC 0697BP) was illustrated in the examples, it is to be understood that a PGA produced by other strains or chemical methods falls within the technical scope of the present invention as long as it is an ultra-high molecular weight PGA with a molecular weight greater than 5,000 kDa.

### EXAMPLE 1

### Production and Molecular Weight Measurement of Ultra-High Molecular Weight PGA

In order to examine if the production of ultra-high molecular weight PGA is made possible through the optimization medium and culturing conditions, the following test was carried out.

A 5L fermenter containing 3L minimal medium (GS medium containing 4% L-glutamate, 3% glucose, 1% (NH₄)₂SO₄, 1% Na-citrate, 0.27% KH₂PO₄, 0.42% Na₂HPO₄, 0.05% NaCl, 0.3% MgSO₄, 1 ml/L vitamin solution, pH 6.8) was inoculated with 1% of a culture broth of Bacillus subtilis var chungkookjang KCTC 0697BP), and cultured at a sting of 150 rpm, an aeration rate of 1 vvm, and 37° C. for 3 days, and then adjusted to pH 3.0 by the addition of 2N sulfuric acid solution, thereby obtaining a PGA-containing sample solution.

The sample solution was left to stand at 4° C. for 10 hours to remove polysaccharides present in the fermented solution, and added with ethanol at the amount of two times volume larger than the fermented solution, and then nixed thoroughly. The mixed solution was left to stand at 4° C. for 10 hours, followed by centrifugation, to give a PGA precipitate.

The precipitate was dissolved by the addition of distilled water, added with 100 μg/ml protease, and allowed to react in a 37° C. incubator, thereby decomposing extracellular protein present in the PGA sample.

The resulting substance was dialyzed against a sufficient amount of distilled water to remove free glutamate, followed by concentration, to give pure PGA.

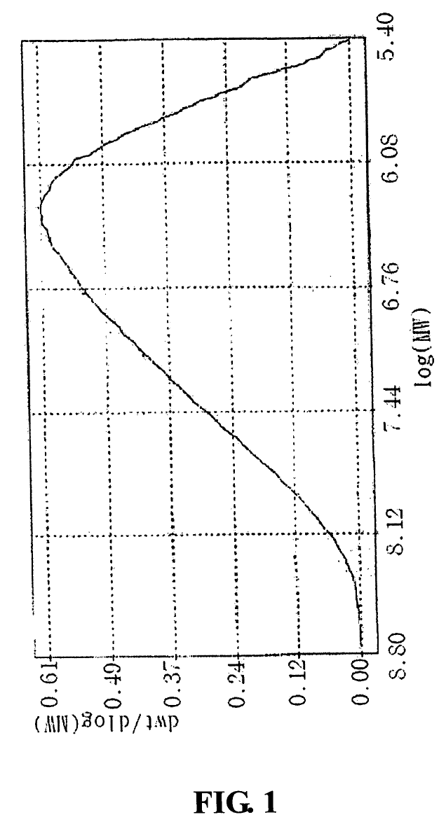

As shown in FIG. 1, it could be found by GPC analysis that the mean molecular weight of the PGA obtained as described above is 13,000 kDa, and more than 95% of its molecules have a molecular weight ranging from 3,000 to 15,000 kDa.

In this case, the molecular weight of the PGA was measured by gel permeation chromatography (GPC). For the molecular weight analysis of PGA using GPC, a GPC system (Youngin Scientific Co, Ltd, Korea) equipped with two GMPW_{XL} columns (VISCOTEK Co.) was used. As a solvent, 0.1N NaNO₃ was used at a flow rate of 0.8 ml/minute. Polyethylene oxide was used as the standard for the GPC analysis, and a refractometer (VISTOTEK Co.) was used to measure the molecular weight of the PGA.

The molecular weight of a prior PGA obtained by the culturing of Bacillus subtilis var. chungkookjang (KCTC 0697BP) was about 2,000 kDa (Korean patent laid-open publication No. 2001-78440), but in the present invention, the ultra-high molecular weight PGA with a molecular weight greater than 5,000 kDa could be successfully produced through the optimization medium and culturing conditions.

### EXAMPLE 2

### Moisture-Absorbing and Moisture-Retaining Properties of Ultra-High Molecular Weight PGA

The moisture-absorbing and moisture-retaining properties of the ultra-high molecular weight PGA produced in Example 1 were compared to an existing PGA having a molecular weight of 600 kDa
(1) Comparison of Moisture-Absorbing Property

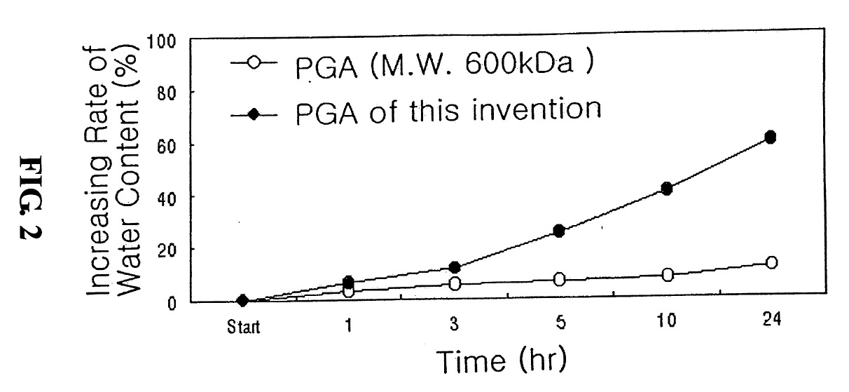

0.5 g of each of the PGA obtained in Example 1 and a prior product with a molecular weight of 600 kDa were put in the respective Petri dish and maintained in a 45° C. incubator for 14 hours to remove water completely. The resulting samples put in a decicator (relative humidity: 81-88%) containing a saturated aqueous solution of calcium carbonate (250 g calcium carbonate per 500 g purified water), and were measured for a change in its weight according to time (moisture-absorbing property) for 24 hours. The measured results are shown in FIG. 2.

As shown in FIG. 2, it was found that the PGA with a 600 kDa molecular weight showed less than 10% increase in water content after 24 hours, whereas the PGA according to the present invention showed about 60% increase in water content, indicating an extraordinarily excellent moisture-absorbing property of the inventive PGA.
(2) Comparison of Moisture-Retaining Property

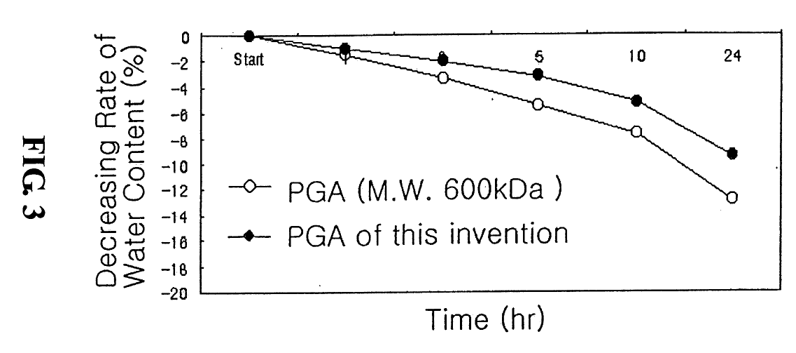

Samples, which had been sufficiently moisturized by standing for 48 hours under the conditions described in the above test (1), were put in a decicator (18% humidity) containing 500 g dry silica gel and measured for a reduction in its water content according to time (moisture-retaining property) for 24 hours at 25° C. The measured results are given in FIG. 3.

As shown in FIG. 3, it was found that the prior PGA with a 600 kDa molecular weight showed 13% reduction in its water content after 24 hours, whereas the ultra-high molecular weight PGA of the present invention showed about 10% reduction in its water content, demonstrating a very excellent moisture-retaining property of the inventive PGA.

From the results of this example, it can be found that the ultra-high molecular weight PGA of the present invention can be used for a variety of moisture-retaining and/or moisture-absorbing products, such as cosmetics, foods, feedstuffs etc.

### Example 3

### Ca Solubility of the Ultra-High Molecular Weight PGA

In order to examine the Ca solubility of the ultra-high molecular weight PGA of the present invention, the following test was carried out.

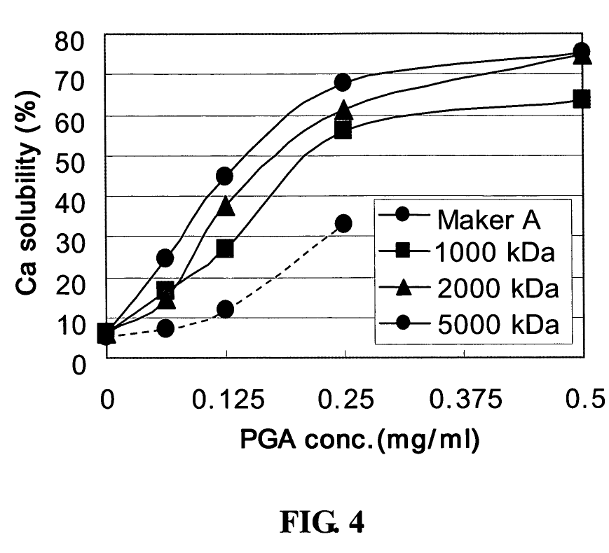

The ultra-high molecular weight PGA produced in Example 1 was diluted to prepare PGA solutions having concentrations of 0.062, 0.125, 0.25 and 0.5 mg/ml, respectively. 0.5 ml of each of the PGA solutions was added to a reaction solution containing 0.5 ml of 10 mM CaCl₂ and 1.0 ml of 20 mM phosphate buffer, followed by reaction at 37° C. After 2 hours, the respective solutions were centrifuged at 2000 g for 30 minutes, and Ca remaining in the supernatant was quantified with a Ca quantification kit (Wako Chemical Co., Japan). In addition, as control groups, a marker A (PGA commercially available from Ajinomoto Co., Japan), a PGA with a molecular weight of 1,000 kDa and a PGA with a molecular weight of 2,000 kDa were tested for their Ca solubility. The test results are shown in FIG. 4.

As shown in FIG. 4, the inventive PGA dissolved (adsorbed) Ca ions at a significantly larger amount than the prior products over all the concentrations. Particularly, at a PGA concentration of 0.125 mg/ml, the marker A, the 1,000-kDa molecular weight PGA and the 2,000-kDa molecular weight PGA showed Ca solubility of about 12%, 27% and 37%, respectively, whereas the ultra-high molecular weight PGA with a 5,000-kDa molecular weight showed a Ca solubility of about 46%.

### Example 4

### Intestinal Ca Absorption-Promoting Effect of Ultra-High Molecular Weight PGA

The ultra-high molecular weight PGA produced in Example 1 was tested for its effect of promoting intestinal Ca absorption.

The PGA with a molecular weight of 5,000 kDa was diluted to prepare solutions having concentrations of 0.05, 0.1 and 0.2%, respectively, and mixed with 5 mM calcium chloride. 1 ml of each of the solutions was administered orally to mice. In order to prove that the ultra-high molecular weight PGA has an excellent effect of promoting intestinal Ca absorption, a comparative test of the inventive PGA and a 1,000-kDa molecular weight PGA was also carried out.

Thirty 4-week-old male BALB/c mice were purchased, housed in a mouse cage under a 12:12-hour dark-light cycle at suitable temperature, and fed with basal feedstuffs and distilled water. The mice were divided into three groups each consisting of 10 animals. The first group was administered with the PGA having a 1,000-kDa molecular weight, the second group was administered with the PGA having a 5,000-kDa molecular weight, and the third group was a control group to which no PGA was administered. The PGA solution sample containing calcium chloride was administered orally to the respective groups, and phosphate buffer solution was administered to the control group.

At 2 hours after oral administration, the animals were anesthetized with ether, and the entire small intestines ranging from the duodenum to the ileum were detached from the abdomen of the mice. The small intestines were divided into two portions of an upper portion and a lower portion, and then washed with cold saline water. Next, the small intestine tissues were homogenized by a homogenizer with the addition of cold saline water. The homogenized tissues were centrifuged at 8,000 rpm and 4° C. for 20 minutes. After centrifugation, a soluble fraction and an insoluble precipitate in the respective tissue samples were collected and stored at −20° C. while analyzing their Ca content with a quantification kit (Wako Chemical Co., Japan). The results of the analysis are given in Table 1 below.

As shown in Table 1, it could be found that the ultra-high molecular weight PGA with a molecular weight of 5,000 kDa showed an excellent effect of promoting Ca absorption. This suggests that the ultrahigh molecular weight PGA can be used for industrial or edible products for Ca absorption.

TABLE 11
Effect of promotion of Ca absorption according to molecular weight of
PGA (Ca content: mg)

| PGA concentration | Upper portion | | Lower portion | |
|---|---|---|---|---|
| (%) | 5,000 kDa | 1,000 kDa | 5,000 kDa | 1,000 kDa |
| 0 | 0.132 | 0.070 | 0.131 | 0.072 |
| 0.05 | 0.147 | 0.075 | 0.134 | 0.074 |
| 0.1 | 0.154 | 0.082 | 0.138 | 0.073 |
| 0.2 | 0.167 | 0.090 | 0.140 | 0.072 |

### Example 5

### Effect of Ultra-High Molecular Weight on Sustained Release of Ca Ions in Intestines

In order to examine if the inventive PGA with a molecular weight of 5,000 kDa has an effect on the sustained release of Ca ions in intestines, the following test was carried out.

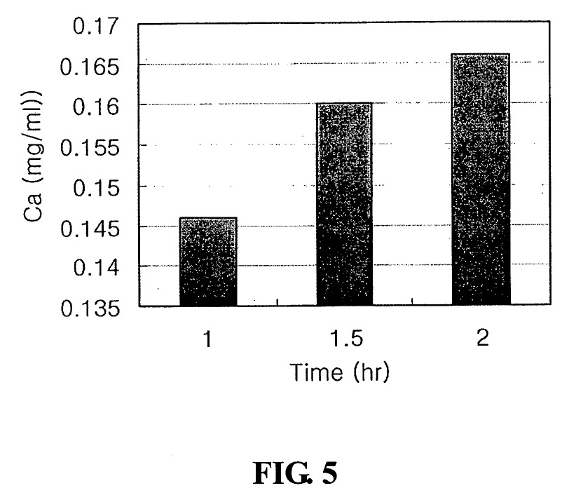

A solution of 0.2% PGA with a molecular weight of 5,000 kDa was mixed with 5 mM calcium chloride, and 1.0 ml of the solution was administered orally to mice. Thereafter, the mice were subjected to the same procedure as in Example 4, except that the mice were anesthetized with ether at 1, 1.5 and 2 after the oral administration of the PGA solution, and then, the entire small intestines ranging from the duodenum to the ileum were detached from the abdomen of the mice. The test results are shown in FIG. 5.

As shown in FIG. 5, the administration of the mixed solution, which contains the inventive PGA having a molecular weight of 5,000 kDa and the calcium chloride, indicated that intestinal Ca absorption rate was increased with the passage of time. This suggests that the PGA according to the present invention has an excellent effect on the sustained release of a mineral in the intestines.

### Example 6

### Effect of Use of Ultra-High Molecular Weight PGA on Promotion of Absorption of Fe Ions into Blood

In order to examine if the use of the inventive PGA with a 5,000-kDa molecular weight has an effect on the promotion of absorption of Fe ions into blood, the following test was conducted.

A solution of 0.04% PGA with a 5,000-kDa molecular weight was mixed with 20 mM ferrous lactate, and 1.0 ml of the solution was administered orally to mice. In order to prove that the ultra-high molecular weight PGA has an excellent effect on the promotion of absorption of Fe ions, a comparative test of the inventive PGA and a 1,000-kDa molecular weight PGA was also carried out.

Thirty 4-week-old male BALB/c mice were purchased, housed in a mouse cage under a 12:12-hour light dark cycle at suitable temperature, and fed with basal feedstuffs and distilled water. The mice were divided into three groups each consisting of 10 animals. The first group was administered with the PGA with a 1,000-kDa molecular weight, the second group was administered with the PGA with a 5,000-kDa molecular weight, and the third group was a control group to which no PGA was administered. The solutions containing the PGA and calcium chloride were administered orally to the respective groups, and the control group was administered with phosphate buffer solution.

At 3 days after oral administration, the animals were anesthetized with ether, and blood was taken from the animals and measured for its Fe content with a particle counter model PCE-170 (ERMA Inc., Japan). The measured Fe content was also expressed in terms of the amount of hemoglobin. The measured results are given in Table 2 below.

As evident from Table 2, it could be found that the administration of the inventive PGA having a 5,000-kDa molecular weight had a very excellent effect on the promotion of Fe absorption into blood. This suggests that the ultra-high molecular weight PGA of the present invention can be used for industrial or edible products for Fe absorption.

TABLE 2
Effect of promotion of Fe absorption according to molecular weight of
PGA

| | | Hemoglobin content | Fe content |
|---|---|---|---|
| No. | Group | (g/100 ml) | (mg/100 ml) |
| 1 | Control group | 12.8-13.1 | 11.1 |
| 2 | PGA (MW: 1,000 kDa) | 14.2-15.3 | 11.9 |
| 3 | PGA (MW: 5,000 kDa) | 14.7-17.0 | 12.8 |

### Example 7

### Water-Absorbing Property of Ultra-High Molecular Weight PGA Hydrogel

5% aqueous solution of each of the ultra-high molecular weight PGA produced in Example 1 and a prior PGA product (600 kDa) was irradiated with gamma ray of 25 kGy, thereby producing hydrogels.

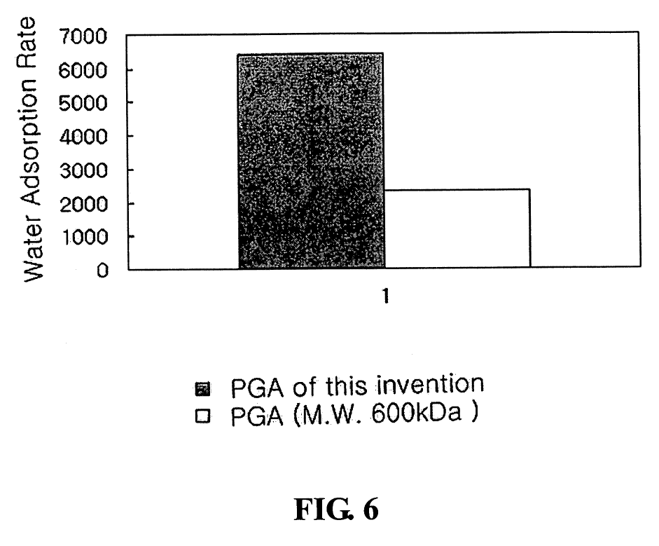

Then, each of the produced hydrogels was immersed in water, and after 24 hours, measured for its weight in water, thereby examining a water-absorbing property of the hydrogels. The measured results are shown in FIG. 6.

As shown in FIG. 6, the prior PGA hydrogel absorbed 2000 times its weight in water, but the inventive PGA hydrogel absorbed 6400 times its weight in water, that indicates 3 times higher water absorption capability than that of the hydrogel containing the prior PGA product. As a result, it can be found that water-absorbing hydrogel produced from the inventive PGA shows an excellent effect of absorbing an increased amount of water even at a lower volume than hydrogel produced from the prior PGA.

### INDUSTRIAL APPLICABILITY

As described above, the present invention provides the ultra-high molecular weight PGA having a molecular weight greater than 5,000 kDa. Furthermore, the present invention provides cosmetics, feedstuffs and foods containing the ultra-high molecular weight PGA, as well as highly water-absorbable hydrogel produced from the ultra-high molecular weight PGA. In addition, the present invention provides the mineral absorption-promoting composition, which contains the ultra-high molecular weight PGA having a molecular weight greater than 5,000 kDa and thus significantly increases the absorption of a mineral into the body. Since the PGA according to the present invention has ultra-high molecular weight, it has very excellent effects on the absorption of a mineral into the body and on the sustained release of a mineral in the body, and thus, can be used for industrial or edible products for mineral absorption.

1. An ultra-high molecular weight poly-gamma-glutamate (PGA) having a mean molecular weight of at least 5,000 kDa.

2. The PGA according to claim 1, which has a mean molecular weight ranging from 5,000 to 15,000 kDa.

3. The PGA according to claim 1, which is produced by Bacillus subtilis var. chungkookjang (KCTC 0697BP).

4. A hydrogel produced from the PGA according to any one of claims 1 to 3.

5. Cosmetics containing the PGA according to any one of claims 1 to 3.

6. Foods containing the PGA according to any one of claims 1 to 3.

7. Feedstuffs containing the PGA according to any one of claims 1 to 3.

8. A water-absorbing agent containing the hydrogel according to claim 4.

9. A mineral absorption-promoting composition, which contains the PGA according to any one of claims 1 to 3, and a mineral.

10. The mineral absorption-promoting composition according to claim 9, which has a sustained release property.

11. The mineral absorption-promoting composition according to claim 9, wherein the mineral is Ca, Fe, Mg, Cu or Se.

12. The mineral absorption-promoting composition according to claim 9, wherein the PGA is substituted with a copolymer of an ultra-high molecular weight PGA having a mean molecular weight of at least 5,000 kDa and a polyamino acid bearing a positive charge.

13. The mineral absorption-promoting composition according to claim 12, wherein the polyamino acid is polylysine or polyarginine.

14. A method for using the PGA according to any one of claims 1 to 3 for a mineral absorption-promoting agent.
